(19) United States
(12) Patent Application Publication
(10) Pub. No.: US 2010/0268191 A1
(43) Pub. Date: Oct. 21, 2010

(54) Drug Delivery Catheter using Frangible Microcapsules and Delivery Method

(75) Inventors: Julie Trudel, Santa Rosa, CA (US); Justin Peterson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/427,177

(22) Filed: Apr. 21, 2009

(51) Int. Cl. A61M 25/10; A61K 9/50

(52) U.S. Cl. 604/509; 604/103.01; 604/101.02

(57) ABSTRACT

A drug delivery catheter and method are provided for delivering drugs to a targeted region of a lumen include drug-laden microcapsules provided within a porous catheter balloon with an effective pore size that prevents free-flow of the microcapsules through the porous wall of the balloon. The microcapsules are frangible under the influence of increased pressure within the balloon. In an alternative embodiment, the microcapsules may be mechanically ruptured by compression between the outer porous balloon and optional, inner, non-porous balloon. The drug is emitted from the microcapsules through the balloon pores and against the targeted luminal surface.

### FIELD OF THE INVENTION

This invention relates to catheters for delivering drugs, pharmacological agents and the like in microcapsule form to a targeted region in a patient and for rupturing the microcapsules to locally release the agent.

### BACKGROUND

The prior art and medical practitioners have long recognized the desirability to deliver drugs or other bioactive or pharmacologically active agents directly to a specific location in the body instead of by systemic delivery. Localized delivery is particularly desirable in vascular applications, for example, to deliver drugs adapted to prevent restenosis as may occur after a percutaneous catheter intervention (PCI) procedure such as angioplasty or stent placement. For example, one such technique is described in U.S. Pat. No. 5,102,402 (Dror) in which a coating of body-affecting chemicals in the form of microcapsules is applied to the exterior of a balloon of a balloon catheter. The coating releases from the balloon when the balloon is inflated into contact with and against a vascular lumen to be treated. Other approaches are described in U.S. Pat. No. 5,580,575 (Unger) and U.S. Pat. No. 7,358,226 (Dayton) that describe drug-carrying microcapsules that can be ruptured by ultrasound to release the drug. Drug-laden microcapsules also have been described as being delivered by direct injection, as in U.S. patent application publication number 2008/0069801 (Lee).

### SUMMARY OF THE INVENTION

It would be desirable for the clinician to receive a simplified arrangement for delivering drug-carrying microcapsules or microcapsules to a targeted region and for releasing the drug at that region. The invention provides an alternate system for delivering biologically active materials in microcapsule form to a specific target location within a patient, such as a particular location within the vascular system. The invention may be practiced, for example, in connection with medications intended to prevent clotting or to deliver agents adapted to prevent restenosis following an angioplasty procedure. The invention is not limited, however, to post-angioplasty applications but may be adapted for use in other vascular or non-vascular applications in appropriate circumstances.

The system includes a delivery catheter having a shaft and a balloon on the distal end of the shaft with an inflation lumen extending through the shaft to communicate with the balloon to permit inflation and deflation. The balloon is porous, having a porous structure including numerous pores with a predetermined maximum effective pore size. A second, internal, non-porous balloon may be disposed, optionally, within the first, outer, porous balloon. The catheter is used together with frangible microcapsules containing the drug, pharmacological or biological agent, the microcapsules being carried in a biocompatible carrier fluid, i.e. in a suspension. The microcapsules are sized to have effective outer dimensions greater than the effective pore size of the balloon so that the microcapsules cannot, by free flow of the suspension, readily pass intact through the pores of the balloon. The materials from which the microcapsules and the balloon are formed are selected so that the microcapsules will deform or rupture sufficiently to release their contents under increased fluid pressure applied to the suspension or by mechanical compression between the outer balloon and the optional inner balloon. The released agent will then be entrained in the fluid that is expelled through the balloon pores. Individual microcapsules may obstruct the pores of the balloon such that, upon their rupture as the microcapsules are forced against the balloon pores, the agent will be ejected directly through the pores and outwardly of the balloon.

In a further embodiment the catheter balloon may be pre-loaded with microcapsules that protect and preserve the drug as well as to enhance the shelf life of the pre-loaded delivery catheter until the intended time of use. The microcapsules may be made from materials selected to be immune to the manufacturing processes, for example, to protect drugs or substances sensitive to sterilization.

The system is used by advancing the catheter to locate the balloon at the intended delivery site. With the balloon in position, inflation fluid (e.g., saline) or a suspension carrying the microcapsules is directed under pressure through the inflation lumen to inflate the balloon against the inner luminal wall of the vessel. The fluid pressure then is increased sufficiently to cause the microcapsules to rupture or deform sufficiently to release their contents, which will be entrained in fluid that is expelled outwardly of the balloon and against the luminal surface of the vessel. The microcapsules are formed from biodegradable materials so that remnants of the microcapsule shells that may be ejected through the pores may dissolve or otherwise break down in the body.

In the dual balloon embodiment of the invention, the outer balloon may be inflated with a microcapsule-carrying suspension to inflate the balloon against the inner luminal surface of the vessel, after which the inner balloon can be inflated to more uniformly redistribute the microcapsules between the balloons to cause more uniform release of the drug from the microcapsules and through the pores of the outer balloon. The inner balloon may be sized to contact an inner surface of the outer balloon to compress and rupture the microcapsules therebetween.

### DESCRIPTION OF THE DRAWINGS

In the accompanying drawings:

FIG. 1 is an illustration of the balloon catheter used in the practice of the invention;

FIG. 2 is an enlarged longitudinal cross-sectional illustration of the catheter of FIG. 1 taken through the region of the balloon;

FIG. 3 is a diagrammatic enlarged transverse sectional illustration of a portion of the balloon wall illustrating the relative dimensions between the pores and the microcapsules;

FIG. 4 is an enlarged illustration of the balloon wall and a microcapsule under pressure and having ruptured to release its contents against the luminal wall of the vessel;

FIG. 5 is a diagrammatic illustration of a sealed package containing a balloon catheter with microcapsules pre-loaded inside the balloon in accordance with an aspect of the invention;

FIG. 6 is an enlarged longitudinal cross-sectional illustration of another balloon catheter used in the practice of the invention with the view taken through the region of the balloon;

FIG. 7 is a diagrammatic enlarged transverse sectional illustration of microcapsules being generally uniformly distributed between the balloon walls of the catheter of FIG. 6; and

FIG. 8 is a diagrammatic enlarged transverse sectional illustration of microcapsules being compressed between the balloon walls of the catheter of FIG. 6.

### DESCRIPTION OF THE ILLUSTRATIVE EMBODIMENTS

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. As used in this specification, the term “drug,” is intended to include any and all biologically active materials usable for diagnosis or therapeutic treatment of the mammalian body. The terms “effective pore size” and “effective microcapsule size” are intended to refer to the relative dimensions of the pores or micropores in the balloon and the drug-carrying microcapsules. The term “fluid” is intended to include gases and liquids although it is preferable to use liquids when the invention is used in the circulatory system. The term “suspension” is intended to mean a mixture of microcapsules dispersed in a fluid.

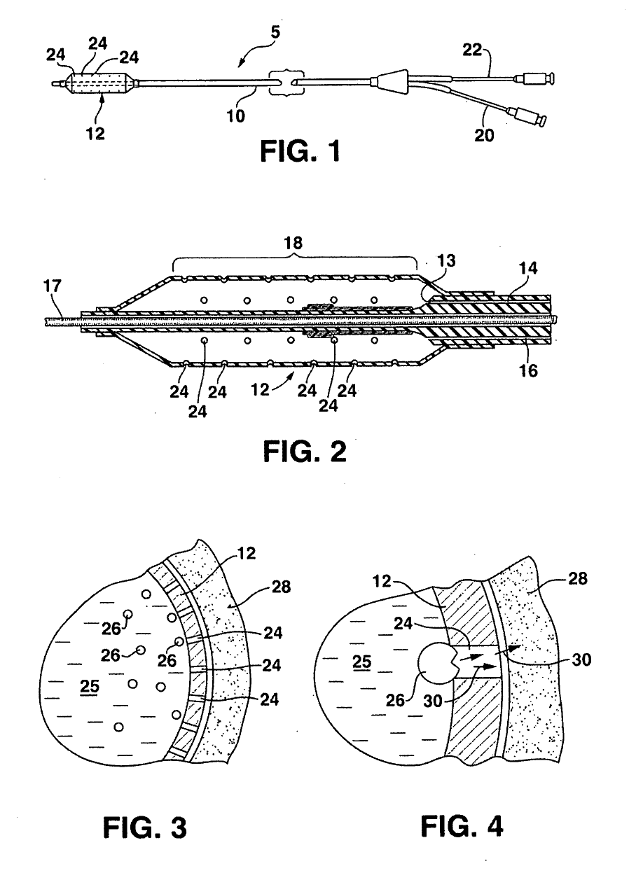

FIGS. 1 and 2 illustrate one embodiment of the invention in the form of a catheter 5 having an elongate shaft 10 with proximal and distal ends. A cylindrical balloon 12 is mounted to the distal end of the catheter shaft 10. The catheter shaft 10 has an inflation lumen 14 that terminates at an inflation port 13 within the balloon and communicates the interior of the balloon 12 with the proximal end of the catheter 5 to enable the balloon to be inflated and deflated. The catheter shaft 10 also may include a guidewire lumen 16 to enable the catheter 5 to be advanced over an indwelling guidewire 17. Catheter shaft 10 is shown as being made from a profile extrusion to provide side-by-side lumens 14, 16, as will be understood by one of ordinary skill in the art of balloon catheters. Other constructions are of course possible, such as a coaxial or tube-in-tube configuration. A pair of tubular tails 20, 22 may be attached to the proximal end of the shaft 10 to communicate with and enable access to the inflation and guidewire lumens 14, 16, respectively.

The catheter shaft 10, fitting 13 and tails 20, 22 may be formed from any of a variety of materials for such components as are well known to those skilled in the art. Balloon 12 should be formed to have a relatively noncompliant construction and may be made, for example, from polyethylene terephthalate (PET), to exhibit little or no stretching when inflated under the pressures sufficient to deform the microcapsules sufficiently to release the drug. The balloon is inflatable, under a relatively low “nominal” pressure, to a nominal diameter at which the balloon will be in apposition with the inner luminal surface of the vessel to which the nominal size of the balloon is adapted. The degree of noncompliance should be selected to assure that the effective pore size of the balloon remains smaller than the effective microcapsule size throughout the range of balloon pressures necessary to release the drug. It should be understood, however, that other balloon materials may be employed provided they have the requisite degree of noncompliance, flexibility and strength sufficient to perform in the manner described herein.

The porous balloon 12 may be fabricated in accordance with U.S. Pat. No. 5,087,244 (Wolinsky), the disclosed processes and materials of which are hereby incorporated by reference in its entirety. Balloon 12 may have a single wall thickness ranging from 0.0002 inches to 0.002 inches. A balloon formed from PET may be fabricated to include a multiplicity of pores 24 that are substantially regularly spaced about a generally cylindrical wall portion 18 of the balloon. The pores 24 may be formed by a variety of techniques including material ablation by an electron beam or by a laser beam from an excimer laser having wavelengths of 248 or 308 nm. As taught in the Wolinsky '244 patent, the aggregate flow area defined by the pores 24 is selected so that under the general range of inflation pressures expected, the liquid flow through the holes will be very low, weeping in nature. The pores 24 should be dimensioned with respect to the microcapsules 26 so that the microcapsules cannot pass freely through the pores, as suggested diagrammatically in FIG. 3, which illustrates the balloon inflated to a pressure at which the microcapsules will be mechanically deformed sufficiently to release the carried drug. For example, the effective microcapsule size may be of the order of about 125% to about 175% the effective pore size of the balloon. It is contemplated that in the practice of the invention microcapsules having an order of five to 100 microns (μ) effective diameter may be employed. It should be understood, however, that the relative effective dimensions of the micropores 24 and the microcapsules 26 are affected by the materials from which the microcapsules are formed as well as the compliance of the balloon 12 and the range of relative effective dimensions may vary accordingly. The effective pore and microcapsule sizes are such as to preclude the unruptured microcapsules from freely passing through the porous balloon wall. The microcapsules of the invention are not necessarily intended to be limited to precisely circular or spherical shapes.

Microcapsules for use in this invention may be made by any of a variety of well-known encapsulating processes using a variety of materials. Among these are those described in U.S. Pat. Nos. 3,516,846; 3,516,941; 3,996,156; 4,087,376; 4,409,156; 5,180,637 and 5,591,146, the disclosed processes and materials of which are hereby incorporated by reference in their entireties. By way of example only, microcapsule walls may be made from natural hydrophilic polymeric materials such as gelatin, gum Arabic, starch, carrageenan and zein; natural polymeric materials may be modified and include ethyl cellulose, carboxymethyl cellulose, shellac resin and nitrous cellulose as well as other polymers including polyvinyl alcohol, polyethylene, polystyrene, polyacrylamide, polyether, polyester, polybutadiene, silicone, epoxy and polyurethane. The materials contained in the microcapsules can be in a variety of forms including solutions, dispersions and gels. These and other materials and processes are described in the references incorporated above. The materials and fabricating processes may be varied and should be selected to produce the combination of microcapsules and balloon pores to cause the microcapsules to be mechanically ruptured sufficiently to cause release of the carried drug out of the balloon and against the vessel wall. As used in this specification the term “rupture” is intended to mean a condition at which the microcapsule has been deformed sufficiently to cause release of the drug from the microcapsule. The fluid pressure in the balloon at which the release may be affected from a particular combination of balloon and microcapsules may be referred to as “rupture pressure.”

FIG. 3 illustrates a segment of the balloon wall of inflated balloon within the lumen of vessel 28 and the manner in which the drug carried by a microcapsule may be delivered to the tissue of the vessel 28 being treated. A microcapsule 26 is shown as being located against or obstructing a pore 24 in the balloon wall with the microcapsule being subjected to increased internal pressure of the inflation fluid 25. FIG. 4 illustrates, in highly diagrammatic form, the rupture of the frangible microcapsule 26 with the drug 30 being emitted directly into pore 24 and outwardly of the balloon 12. It should be understood that the microcapsules may rupture or collapse in a variety of modes that result in the ejection of drugs through the pores, depending on the materials and structure of the balloon, microcapsules and the rupture pressure. Microcapsules 26 may be ruptured in response to increased internal pressure of inflation fluid 25 without the microcapsules being located against the balloon wall or obstructing any pores 24. In such cases, the released drug or agent will be entrained in fluid 25 that is expelled through balloon pores 24 against vessel 28.

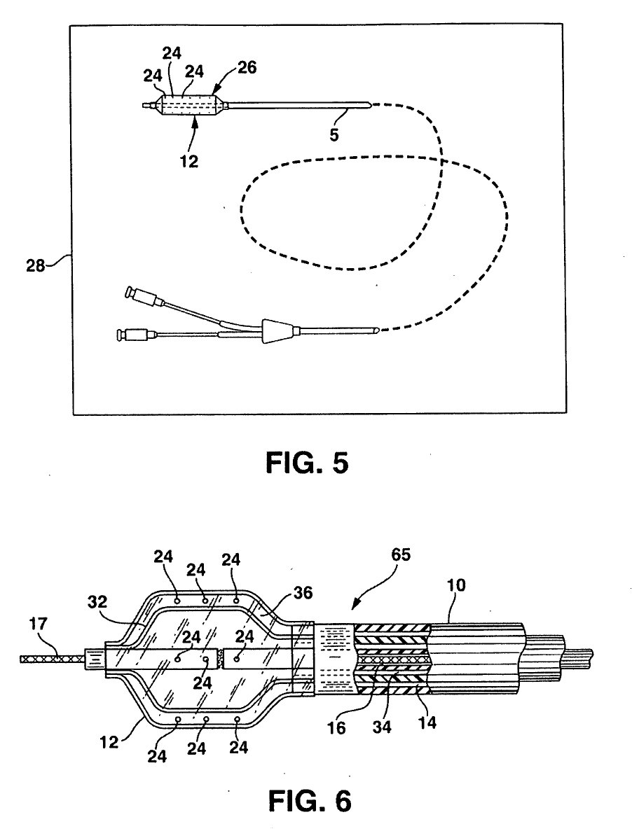

FIG. 5 illustrates a drug delivery system in accordance with the invention wherein, prior to packaging the catheter 5, microcapsules 26 have been pre-loaded into. porous balloon 12 using, for example, a dry process such as insufflation. Pouch-type package 28 is sealed to maintain sterility of the catheter 5 and microcapsules 26 contained there within. During preparation for use, a catheter 5 containing pre-loaded microcapsules may be filled with sterile inflation fluid 25 to flush air from the balloon 12 and inflation lumen 20 and to create a microcapsule suspension. After flushing and before the catheter 5 is inserted into the patient, balloon 12 may be deflated in a sterile saline bath to prevent aspiration of air into the catheter 5.

Microcapsules 26 may be received by the clinician in various ways for use in the invention. For example, the microcapsules 26 may be received dry and pre-loaded within porous balloon 12. Alternatively, a vial containing either dry or suspended microcapsules may be received within package 28 or separately therefrom. Dry microcapsules may be mixed with suitable fluid either within balloon 12 or outside the catheter 5 to prepare a suspension for use as described herein. In accordance with the invention, microcapsules 26 may be stored, handled, mixed with fluids and/or injected into catheter 5 before finally being caused to rupture within balloon 12 and thereby release the contained drug or agent for ejection through pores 24.

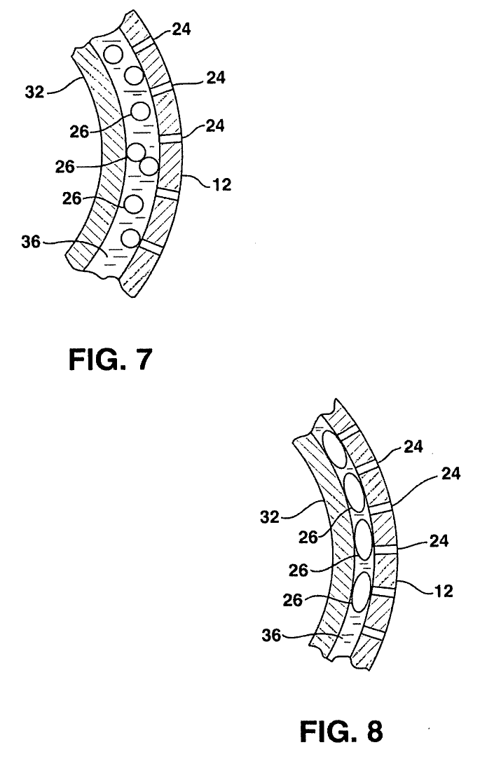

FIGS. 6 and 7 illustrate another embodiment of the invention in which the delivery catheter 65 includes a second, internal non-porous balloon 32 having a separate second inflation lumen 34 extending through the shaft 10. The inflated dimensions of the inner balloon 32 may be less than those of the outer balloon 12 to form an annular chamber 36 adapted to receive and contain the microcapsule suspension and to distribute the microcapsules 26 substantially uniformly within balloon 12. Catheter 65 is constructed of several nested coaxial tubes to create inner guidewire lumen 16, inner inflation lumen 34 and outer inflation lumen 14, as will be understood by one of ordinary skill in the art of balloon catheters. Other constructions are of course possible, such as a three-lumen profile extrusion.

Alternatively, the second balloon 32 has an outer diameter sized for being inflated into contact with an inner surface of the generally cylindrical wall portion 18 of the first balloon 12. First and second balloons 12, 32 are configured by size and material properties to be capable of rupturing microcapsules 26 by mechanically compressing the microcapsules between the first and second balloons in response to the inflation pressure within inner balloon 32, as illustrated in FIG. 8.

It should be understood that the foregoing description of the invention is intended to be merely illustrative only and that other embodiments, modifications and equivalents within the scope of the invention may be apparent to those skilled in the art.

1. A device for delivering drugs locally to a target site in a mammalian body, comprising:
a catheter having an elongate shaft with proximal and distal ends and an inflation lumen extending therethrough; and
a relatively noncompliant balloon mounted at a fixed location to and about a distal region of the shaft, the interior of the balloon in communication with the inflation lumen and being inflatable to a nominal outer diameter by fluid at a nominal balloon pressure, the balloon having a generally cylindrical relatively noncompliant wall portion with a multiplicity of pores therethrough, the pores having a predetermined effective pore size; and
a multiplicity of drug-laden microcapsules contained within the interior of the balloon and having a predetermined effective microcapsule size that is greater than the predetermined effective size of the pores in the balloon wall, whereby microcapsules contained within the balloon are unable to pass intact through the pores, the microcapsules being mechanically frangible under the influence of a predetermined rupture pressure that is greater than the nominal balloon pressure;
whereby, microcapsules contained within the balloon may be ruptured by application of rupture pressure applied to the interior of the balloon to release the drug and enable it to be delivered through the pores of the balloon.

2. A device as defined in claim 1 wherein the balloon is sufficiently non-compliant so that the effective pore size does not expand beyond the effective size of the microcapsules upon application of rupture pressure.

3. A device as defined in claim 2 wherein the balloon is formed from polyethylene terephthalate.

4. A device as defined in claim 1 wherein the microcapsules have an effective size of between about 5 to about 100 microns.

5. A device as defined in claim 1 wherein the effective size of the microcapsules is about 125% to about 175% of the effective pore size.

6. A device as defined in claim 1 further comprising a sealed package containing the balloon catheter having the drug-laden microcapsules disposed within the balloon.

7. A device as defined in claim 1 further comprising:
a second non-compliant, non-porous balloon mounted at a fixed location to and about the catheter shaft within the porous balloon to define between the porous and second balloons a chamber adapted to receive a suspension containing the microcapsules; and
a second inflation lumen extending through the shaft and in communication with the second balloon to provide for selective inflation thereof.

8. A device as defined in claim 7 wherein the second balloon has an outer diameter capable of being inflated into contact with an inner surface of the generally cylindrical wall portion of the porous balloon.

9. A method for delivering drugs locally to a target site in a mammalian body, the method comprising:
receiving a catheter having an elongate shaft with proximal and distal ends and an inflation lumen extending therethrough and a relatively noncompliant balloon mounted at a fixed location to and about a distal region of the shaft, the interior of the balloon being in communication with the inflation lumen and being inflatable to a nominal outer diameter by fluid at a nominal balloon pressure, the balloon having a generally cylindrical relatively noncompliant wall portion with a multiplicity of pores therethrough, the pores having a predetermined effective pore size;
receiving a multiplicity of drug-laden microcapsules, having an effective microcapsule size that is greater than the predetermined effective size of the pores in the balloon wall and being frangible at a predetermined rupture pressure greater than that required to inflate the first balloon to a nominal size;
if the microcapsules are not in within the balloon, positioning the drug-carrying microcapsules within the balloon;
advancing the catheter through a lumen of the body and positioning the balloon at the target site;
inflating the balloon with a fluid inflation medium to the balloon nominal size in apposition with the target site; and
rupturing the microcapsules to release the drug through the pores of the balloon.

10. The method as defined in claim 9 wherein the steps of inflating the balloon and positioning the drug-carrying microcapsules within the balloon are carried out simultaneously after positioning the balloon at the target site using a suspension containing the microcapsules.

11. The method as defined in claim 10 wherein the step of inflating the balloon causes the microcapsules to obstruct fluid flow through the pores.

12. The method as defined in claim 9 wherein the step of rupturing the microcapsules further comprises increasing the pressure in the balloon to a pressure greater than the predetermined rupture pressure of the microcapsules.

13. The method as defined in claim 9 wherein the catheter further has a second non-porous non-compliant balloon mounted about the catheter shaft within the first porous balloon to define an annular chamber between the porous and second balloons to contain the microcapsules.

14. The method as defined in claim 13 further comprising inflating the second balloon after inflating the porous balloon to distribute the microcapsules substantially uniformly within the chamber.

15. The method as defined in claim 14 wherein the step of rupturing of the microcapsules further comprises mechanically compressing the microcapsules between the porous and second balloons.

16. The method as defined in claim 13 wherein the second balloon has an outer diameter capable of being inflated into contact with an inner surface of the generally cylindrical wall portion of the porous balloon.

17. A method for delivering drugs locally to a target site in a mammalian body, the method comprising:
receiving a catheter having an elongate shaft with proximal and distal ends and an inflation lumen extending therethrough and a relatively noncompliant balloon mounted at a fixed location to and about a distal region of the shaft, the interior of the balloon being in communication with the inflation lumen and being inflatable to a nominal outer diameter by fluid at a nominal balloon pressure, the balloon having a generally cylindrical relatively noncompliant wall with a multiplicity of pores therethrough, the pores having a predetermined effective pore size; and a multiplicity of drug-laden microcapsules contained within the interior of the balloon and having a predetermined effective microcapsule size that is greater than the predetermined effective size of the pores in the balloon wall, whereby microcapsules contained within the balloon are unable to pass intact through the pores, the microcapsules being mechanically frangible under the influence of a predetermined rupture pressure that is greater than the nominal balloon pressure;
advancing the catheter through a lumen of the body and positioning the first balloon at the target site;
inflating the balloon with a fluid inflation medium to the balloon nominal size in apposition with the target site; and
rupturing the microcapsules to release the drug through the pores.
